(19) United States
(12) Patent Application Publication
(10) Pub. No.: US 2011/0245292 A1
(43) Pub. Date: Oct. 6, 2011

(54) 2-HYDROXY-ETHANESULFONATE SALT

(75) Inventor: Alan John Nadin, Essex (GB)

(73) Assignees: AstraZeneca AB, Soedertaelje (SE); Pulmagen Therapeutics (synergy) Limited, Harlow, Essex (GB)

(21) Appl. No.: 13/058,773

(22) PCT Filed: Aug. 11, 2009
(86) PCT No.: PCT/SE09/50926

§ 371 Date: Jun. 23, 2011

(30) Foreign Application Priority Data
Aug. 12, 2008 (GB) 0814728.2
Aug. 12, 2008 (GB) 0814729.0

(51) Int. Cl. A61K 31/439; C07D 453/02; A61P 11/00

(52) U.S. Cl. 514/305; 546/137

(57) ABSTRACT

The invention provides (R)-1-[3-(R)-cyclohexyl-hydroxy-phenyl-methyl)-isoxazol-5-ylmethyl]-3-(3-fluoro-phenoxy)-1-azonia-bicyclo[2.2.2]octane 2-hydroxy ethanesulfonate, pharmaceutical compositions containing the compound and its use as a muscarinic antagonists, for the treatment of chronic obstructive pulmonary disease.

The present invention relates to a salt of a muscarinic antagonist, a pharmaceutical composition containing it and its use in therapy.

Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue. Parasympathetic nerves are the main pathway for reflex bronchoconstriction in human airways and mediate airway tone by releasing acetylcholine onto muscarinic receptors. Airway tone is increased in patients with respiratory disorders such as asthma and chronic obstructive pulmonary disease (COPD), and for this reason muscarinic receptor antagonists have been developed for use in treating airway diseases. Muscarinic receptor antagonsists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1, 223-229).

When used to treat respiratory disorders, muscarinic receptor antagonists are typically administered by inhalation. However, when administered by inhalation a significant proportion of the muscarinic receptor antagonist is often absorbed into the systemic circulation resulting in reported side effects such as dry mouth. Additionally, the majority of muscarinic antagonists have a relatively short duration of action requiring that they be administered several times a day. Such a multiple-daily dosing regime is not only inconvenient to the patient but also creates a significant risk of inadequate treatment due to patient non-compliance associated with the frequent repeat dosing schedule. There therefore remains a need for novel compounds that are capable of blocking muscarinic receptors. In particular, a need exists for new muscarinic antagonists that have high potency and reduced systemic side effects when administered by inhalation. Moreover, a need exists for new muscarinic antagonists that exhibit a long duration of action when dosed by inhalation, and which are amenable to either once or twice daily dosing.

In the manufacture of pharmaceutical formulations, it is important that the active compound is in a form in which it can be conveniently handled and processed in order to obtain a commercially-viable manufacturing process. In this connection, the chemical stability and the physical stability of the active compound are important factors. The active compound, and formulations containing it, must be capable of being effectively stored over appreciable periods of time, without exhibiting any significant change in the physico-chemical characteristics (e.g. chemical composition, density, hygroscopicity and solubility) of the active compound.

Furthermore, if the active compound is to be incorporated into a formulation for pulmonary administration, it is desirable if the active compound can be readily micronised to yield a powder with good flow properties and comprising a high fine crystalline particle fraction (i.e. a fraction in which the active compound particles have a mass median aerodynamic diameter of less than 10 μm (micrometer)). Such a fraction is capable of being carried deep into the lungs leading to faster and increased absorption of the active compound.

International Patent Application WO2008/099186 (PCT/GB2008/000519) describes a novel class of muscarinic antagonist that display high potency to the M3 receptor. One such muscarinic antagonist described in PCT/GB2008/000519 is (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane chloride. However, the chloride salt described is hygroscopic and poorly crystalline. It has now been found possible to prepare an alternative salt of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane which has good physico-chemical properties and which may be suitable for use in a dry powder formulation for pulmonary administration.

Thus, in accordance with the present invention, there is provided a salt which is a 2-hydroxy-ethanesulfonate salt of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane.

The salt of the present invention is herein referred to as (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate. The name (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane is a IUPAC name generated by the Beilstein Autonom 2000 naming package, as supplied by MDL Information Systems Inc., based on the structures depicted in Figure A, and stereochemistry assigned according to the Cahn-Ingold-Prelog system.

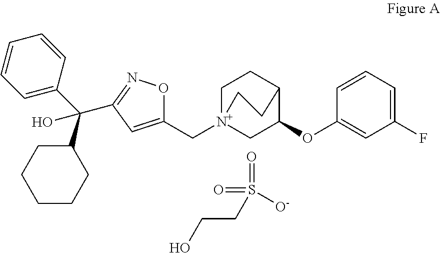

In an embodiment of the invention, the salt has crystalline properties and is at least 50% crystalline. In a further embodiment, the salt is at least 60% crystalline; in a still further embodiment at least 70% crystalline and in a yet further embodiment at least 80% crystalline. Crystallinity can be estimated by conventional X-ray diffractometry techniques.

In another embodiment of the invention, the salt is from 50%, 60%, 70%, 80% or 90% to 95%, 96%, 97%, 98%, 99% or 100% crystalline.

In one embodiment, the stoichiometric ratio of cation to anion in the salt of the present invention is approximately 1:1, i.e. in the range of from 1:0.9 to 1:1.

An example of a crystalline form of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate is crystalline Form A as defined herein below. Thus, in one embodiment the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic X-ray powder diffraction peaks (expressed in degrees 20 when using λ=1.5418): 8.4, 14.7 and 16.8.

In a further embodiment, the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic X-ray powder diffraction peaks (expressed in degrees 20 when using λ=1.5418): 8.4, 14.7, 16.8 and 25.3.

In a further embodiment, the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic X-ray powder diffraction peaks (expressed in degrees 20 when using λ=1.5418): 8.4, 14.7, 16.8, 18.9 and 25.3.

In a further embodiment, the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic X-ray powder diffraction peaks (expressed in degrees 20 when using λ=1.5418): 8.4, 11.8, 14.7, 16.8, 18.9 and 25.3.

In a further embodiment, the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic X-ray powder diffraction peaks (expressed in degrees 20 when using λ=1.5418): 8.4, 11.8, 14.7, 16.8, 18.9, 23.7 and 25.3.

In a further embodiment, the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic X-ray powder diffraction peaks (expressed in degrees 20 when using λ=1.5418): 8.4, 11.8, 12.3, 14.7, 16.8, 18.9, 23.7 and 25.3.

In the present specification unless otherwise stated the margin of error for X-ray powder diffraction peaks (expressed in degrees 20) is consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089). In an embodiment of the invention, the margin of error for X-ray powder diffraction peaks (expressed in degrees 20) is (±0.1°).

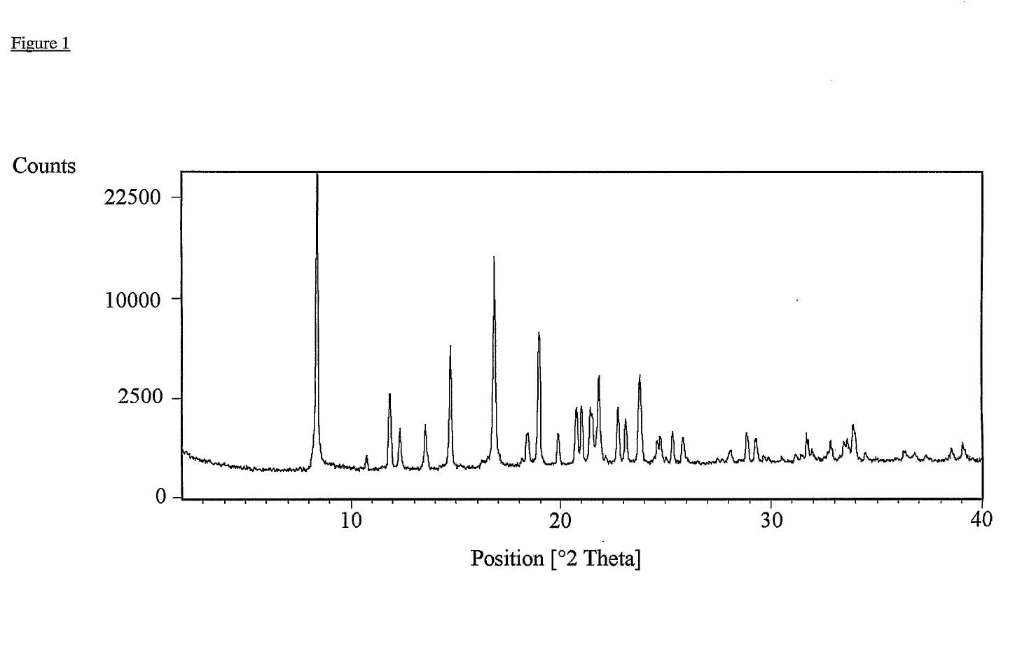

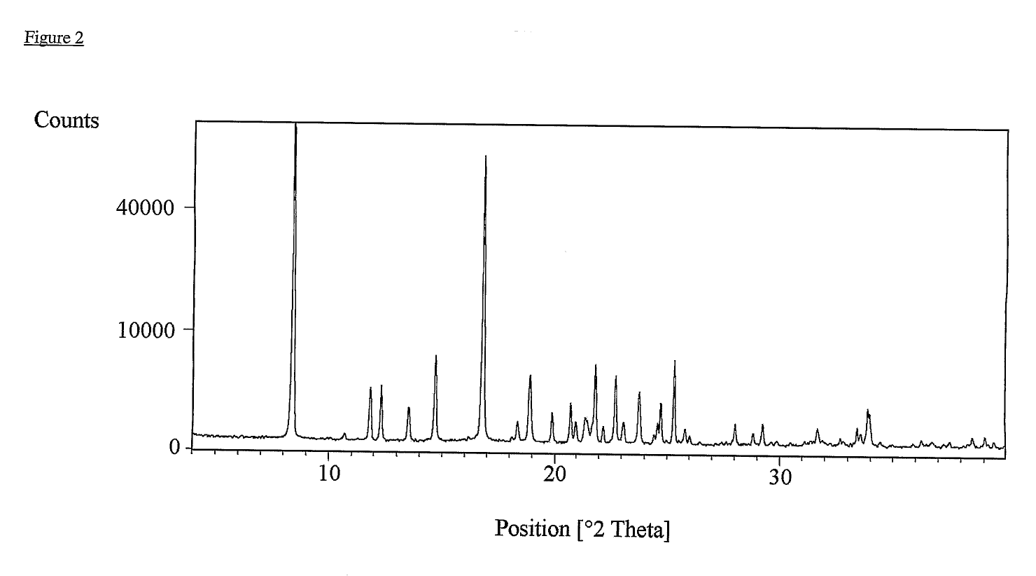

FIGS. 1 and 2 show X-ray powder diffraction patterns of Salt Form A of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

The present invention further provides a salt form having an X-ray powder diffraction pattern substantially the same as that shown in FIG. 1. The X-ray powder diffraction pattern of FIG. 2 is substantially the same as that of FIG. 1.

In one embodiment the present invention provides a salt form (Salt Form A) of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate which exhibits at least the following characteristic d-space values:
(1) 10.5, 6.0 and 5.3, or
(2) 10.5, 6.0, 5.3 and 3.5, or
(3) 10.5, 6.0, 5.3, 4.7 and 3.5, or
(4) 10.5, 7.5, 6.0, 5.3, 4.7 and 3.5, or
(5) 10.5, 7.5, 6.0, 5.3, 4.7, 3.7 and 3.5, or
(6) 10.5, 7.5, 7.2, 6.0, 5.3, 4.7, 3.7 and 3.5.

In an embodiment of the invention, Salt Form A is an anhydrate (i.e. a crystalline phase that does not contain water). In an embodiment of the invention, Salt Form A has a water uptake value of less than 1% as measured by the increase in mass determined by GVS at 80% relative humidity and 25° C.

An embodiment of the invention provides Salt Form A substantially free of other physical forms. Substantially free of other physical forms means that at least 90% by weight, e.g. 90, 91, 92, 93, 94, 95, 96, 97, 98 or 100% of the salt is in that physical form.

(R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate may be prepared from (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane chloride using anion exchange techniques. For example, by preparing a solution of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane chloride in a suitable solvent (e.g. dichloromethane), mixing said solution with an aqueous solution of ammonium isoethionate at a suitable temperature (e.g. 0 to 50° C.), and then isolating (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate from the mixture. Specific details of a preparation of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate are given herein below in the examples.

The salt of the invention has activity as a pharmaceutical, in particular as an anticholinergic agent including a muscarinic receptor (M1, M2, and M3) antagonist, in particular a M3 antagonist. Diseases and conditions which may be treated with the salt include:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvovaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS. Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,

15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention further provides (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate as hereinbefore defined for use in therapy.

In another aspect, the invention provides the use of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate as hereinbefore defined, in the manufacture of a medicament for use in therapy.

In the context of the present specification, the term “therapy” also includes “prophylaxis” unless there are specific indications to the contrary. The terms “therapeutic” and “therapeutically” should be construed accordingly.

A further aspect of the invention provides a method of treating a disease state in a mammal suffering from, or at risk of, said disease, which comprises administering to a mammal in need of such treatment a therapeutically effective amount of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate as hereinbefore defined.

The present invention also provides (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate for use in the treatment of chronic obstructive pulmonary disease (COPD) (such as irreversible COPD).

The present invention also provides the use of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate as hereinbefore defined, in the manufacture of a medicament for use in the treatment of chronic obstructive pulmonary disease (COPD) (such as irreversible COPD).

The present invention also provides the use of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate as hereinbefore defined, in the manufacture of a medicament for use in the treatment of asthma.

The present invention further provides a method of treating chronic obstructive pulmonary disease (COPD) (such as irreversible COPD), in a warm-blooded animal, such as man, which comprises administering to a mammal in need of such treatment an effective amount of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate as hereinbefore defined.

In order to use a compound of the invention for the therapeutic treatment of a warm-blooded animal, such as man, said ingredient is normally formulated in accordance with standard pharmaceutical practice as a pharmaceutical composition.

For the above-mentioned therapeutic uses the dosage administered will, of course, vary with the mode of administration, the treatment desired and the disorder indicated but may typically be in the range from 0.001 mg/kg to 30 mg/kg.

The salt according to the invention may be used on its own but will generally be administered in the form of a pharmaceutical composition in which the (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition may comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate in association with a pharmaceutically acceptable adjuvant, diluent or carrier.

The invention further provides a process for the preparation of a pharmaceutical composition of the invention which comprises mixing (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

In an embodiment of the invention, the active ingredient is administered by inhalation. In a further embodiment, the active ingredient is administered by means of a dry powder inhaler. The inhaler may be a single or a multi dose inhaler, and may be a breath actuated dry powder inhaler.

When administered via inhalation the dose of the active ingredient may generally be in the range of from 0.1 μg to 10000 μg, 0.1 to 5000 μg, 0.1 to 1000 μg, 0.1 to 500 μg, 0.1 to 200 μg, 0.1 to 200 μg, 0.1 to 100 μg, 0.1 to 50 μg, 5 μg to 5000 μg, 5 to 1000 μg, 5 to 500 μg, 5 to 200 μg, 5 to 100 μg, 5 to 50 μg, 10 to 5000 μg, 10 to 1000 μg, 10 to 500 μg, 10 to 200 μg, 10 to 100 μg, 10 to 50 μg, 20 to 5000 μg, 20 to 1000 μg, 20 to 500 μg, 20 to 200 μg, 20 to 100 μg, 20 to 50 μg, 50 to 5000 μg, 50 to 1000 μg, 50 to 500 μg, 50 to 200 μg, 50 to 100 μg, 100 to 5000 μg, 100 to 1000 μg or 100 to 500 μg.

Dry powder formulations and pressurized HFA aerosols of the active ingredient may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The invention will now be illustrated by the following non-limiting Examples. In the Examples the following Figures are presented:

FIG. 1: X-ray powder diffraction pattern of Salt Form A of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate prepared in preparation 1.

FIG. 2: X-ray powder diffraction pattern of Salt Form A of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate prepared in preparation 2.

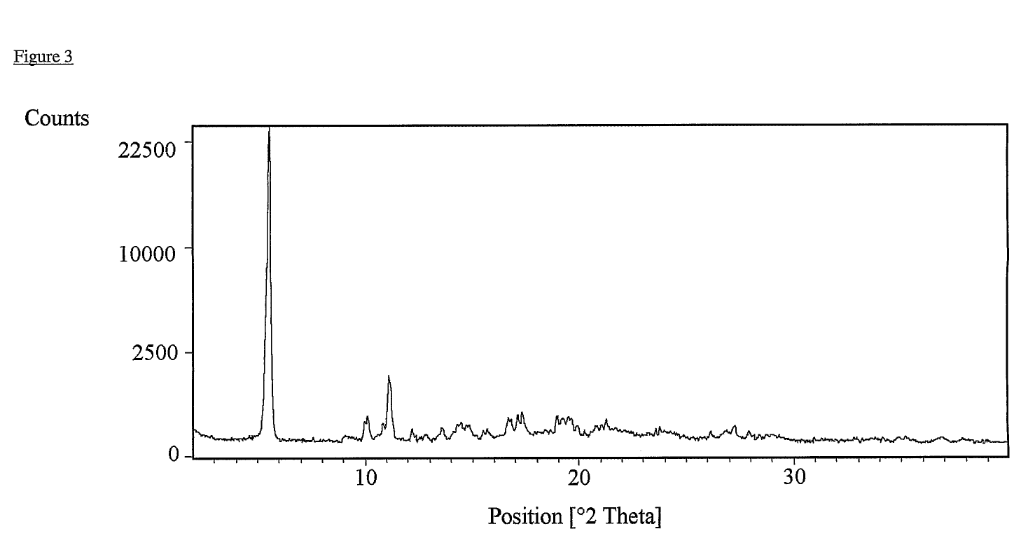

FIG. 3: X-ray powder diffraction pattern of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane chloride.

### GENERAL EXPERIMENTAL DETAILS

All reactions were carried out under an atmosphere of nitrogen unless specified otherwise. NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz. Shifts are given in ppm relative to tetramethylsilane. Where products were purified by column chromatography, ‘flash silica’ refers to silica gel for chromatography, 0.035 to 0.070 mm (220 to 440 mesh) (e.g. Fluka silica gel 60), and an applied pressure of nitrogen up to 10 p.s.i accelerated column elution or use of the semi-automated CombiFlash® Companion purification system or by manual elution of Biotage® Isolute Flash Si II cartridges under reduced pressure or by use of the Biotage® SP1 semi-automated system. All solvents and commercial reagents were used as received. SCX chromatography was performed on Biotage® Isolute SCX or SCX-2 pre-packed cartridges.

The Liquid Chromatography Mass Spectroscopy (LCMS) methods referred to are described below:

Method 1

Waters Micromass ZQ2000 with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection—MS, ELS, UV (100 μl split to MS with in-line UV detector) MS ionisation method—Electrospray (positive ion)

Method 2

Waters Platform LC Quadrupole mass spectrometer with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (200 μA split to MS with in-line UV detector) MS ionisation method—Electrospray (positive and negative ion).

Abbreviations used in the experimental section: DCM=dichloromethane; DMF=dimethylformamide; DMSO=dimethyl sulfoxide; IMS=industrial methylated spirit; LCMS=Liquid Chromatography-Mass Spectrometry; NBS=N-bromosuccinimide; RT=room temperature; Rt=retention time; TFA=trifluoroacetic acid; THF=tetrahydrofuran; SCX=strong cation exchange chromatography.

Preparation of Intermediates

### (R)-3-(3-Fluoro-phenoxy)-1-aza-bicyclo[2.2.2]octane—Intermediate 1

A solution of (R)-1-aza-bicyclo[2.2.2]octan-3-ol (1.25 g), CuI (93.1 mg), 1,10-phenanthroline (176 mg), Cs₂CO₃ (3.19 g) and 3-fluoro-iodo-benzene (1.11 g) in toluene (2.5 mL) was heated at 100° C. for 20 h. The reaction mixture was cooled, diluted with ethyl acetate and filtered through Celite. The insoluble material was washed several times with ethyl acetate. The filtrate was washed with 5% copper sulphate solution, water, dried (MgSO₄), filtered and evaporated in vacuo. Purification by SCX gave (R)-3-(3-fluoro-phenoxy)-1-aza-bicyclo[2.2.2]octane (490 mg, 45%) as a brown oil. LCMS (Method 2, Rt 2.09 min). MH⁺=222.

### (R)-(5-Chloromethyl-isoxazol-3-yl)-cyclohexyl-phenyl-methanol—Intermediate 2

Step 1: 1,1′-Carbonyl diimidazole (25.0 g, 154 mmol) was added to a stirred suspension of (R)-cyclohexyl-hydroxy-phenyl-acetic acid (30.0 g, 128 mmol) in dry THF (600 mL). After stirring for 90 mins at room temperature, sodium borohydride (11.6 g, 307 mmol) was added portionwise over a period of 1 hour. The reaction mixture was then left to stir at room temperature overnight. The reaction was quenched by the addition of water (100 mL) then extracted with DCM. The combined organic phases were dried (MgSO₄), filtered and evaporated in vacuo to give a crude solid. Purification by silica gel chromatography (eluting with 0-5% methanol in DCM) gave (R)-1-cyclohexyl-1-phenyl-ethane-1,2-diol (20.7 g, 73%). ¹H NMR (400 MHz, CDCl₃): δ 7.41-7.33 (4H, m), 7.28-7.24 (1H, m), 3.99 (1H, d), 3.83 (1H, d), 2.68 (1H, br s), 1.86-1.80 (1H, m), 1.78-1.64 (3H, m), 1.63-1.57 (1H, m), 1.47-1.41 (1H, m), 1.27-0.94 (5H, m).

Step 2: A solution of oxalyl chloride (15.5 mL, 201 mmol) in dry DCM (900 mL) was cooled to −78° C. under a nitrogen atmosphere. A solution of DMSO (28.5 mL, 401 mmol) in DCM (25 mL) was added dropwise then the mixture stirred at −78° C. for 10 mins. A solution of (R)-1-cyclohexyl-1-phenyl-ethane-1,2-diol (29.5 g, 134 mmol) in DCM (250 mL) was added dropwise over the course of 1 hour giving a thick slurry. The internal temperature was allowed to reach −45° C. Triethylamine (92.8 mL, 669 mmol) was added dropwise and after complete addition the mixture was allowed to warm to room temperature. The mixture was washed with 1N hydrochloric acid (500 mL×2), water (500 mL) and brine (500 mL) then dried (MgSO₄), filtered and evaporated to give an orange-coloured oil. This was dissolved in IMS (320 mL) and added portionwise to a preformed solution of hydroxylamine hydrochloride (14.0 g, 201 mmol) and sodium carbonate (21.3 g, 201 mmol) in water (210 mL). The resulting emulsion was stirred at room temperature overnight then partitioned between DCM and water. The organic layer was washed with water and brine, then dried (MgSO₄), filtered and evaporated in vacuo. Purification by silica gel chromatography (eluting with 0-15% EtOAc in cyclohexane) gave (R)-cyclohexyl-hydroxy-phenyl-acetaldehyde oxime (25.9 g, 83%). ¹H NMR (400 MHz, CDCl₃): δ 7.76 (1H, s), 7.44-7.41 (2H, m), 7.37-7.33 (2H, m), 7.27-7.23 (1H, m), 7.22 (1H, br s), 3.34 (1H, s), 1.90-1.60 (5H, m), 1.37-1.05 (6H, m).

Step 3: A solution of (R)-cyclohexyl-hydroxy-phenyl-acetaldehyde oxime (8 g, 34 mmol) and 2,6-lutidine (10 mL, 86 mmol) in DCM (150 mL) was cooled in an ice-bath. Trimethylsilyl trifluoromethanesulfonate (15.6 mL, 86 mmol) was added dropwise. The mixture was stirred for 10 minutes at 0° C. then allowed to warm to room temperature for 30 mins. The reaction was quenched by addition of water (50 mL). The organic phase was isolated by passage though a phase separation cartridge and evaporated in vacuo. Purification by silica gel chromatography (eluting with 10-20% EtOAc in cyclohexane) gave a mixture of mono and bis TMS-protected compounds. This was dissolved in methanol and left at room temperature overnight and evaporated in vacuo to give (R)-cyclohexyl-phenyl-trimethylsilanyloxy-acetaldehyde oxime (10 g, 96%). ¹H NMR (400 MHz, CDCl₃): δ 7.62 (1H, s), 7.32-7.28 (4H, m), 7.26-7.21 (1H, m), 7.11 (1H, s), 1.93-1.85 (2H, m), 1.76-1.71 (1H, m), 1.68-1.56 (2H, m), 1.49-1.42 (1H, m), 1.27-0.78 (5H, m), 0.11 (9H, m).

Step 4: A solution of (R)-cyclohexyl-phenyl-trimethylsilanyloxy-acetaldehyde oxime (6 g, 19.6 mmol) was formed in dry DCM (400 mL) and cooled to −78° C. Under reduced lighting, a solution of tert-butylhypochlorite (4.3 g, 39.3 mmol) in DCM (10 mL) was added dropwise. After 2 hours at −78° C. a solution of triethylamine (4.1 mL, 29.4 mmol) in DCM (10 mL) was added dropwise. After a further 10 mins at −78° C. the mixture was allowed to warm to 0° C. At this point, propargyl chloride (14.4 mL, 196 mmol) was added and the mixture was allowed to warm to room temperature overnight. The mixture was washed with brine (200 mL), dried (Na₂SO₄), filtered and evaporated. Purification by silica gel chromatography (eluting with 0-10% EtOAc in cyclohexane) gave crude 5-chloromethyl-3-((R)-cyclohexyl-phenyl-trimethylsilanyloxy-methyl)-isoxazole. This was re-dissolved in THF (100 mL), cooled in an ice-bath and a solution of tetrabutylammonium fluoride (19.6 mL of 1 M in THF) was added dropwise. This mixture was stirred for 30 mins at 0° C. then partitioned between ethyl acetate and water. The organic phase was dried (Na₂SO₄), filtered and evaporated in vacuo. Purification by silica gel chromatography (eluting with 0-20% EtOAc in cyclohexane) gave the title compound as a white solid (3.5 g, 58%). ¹H NMR (400 MHz, CDCl₃): δ 7.51 (2H, m), 7.32 (2H, m), 7.25-7.21 (1H, m), 6.29 (1H, s), 4.52 (2H, s), 2.80 (1H, s), 2.34-2.28 (1H, m), 1.81-1.76 (1H, m), 1.72-1.62 (3H, m), 1.36-1.02 (6H, m).

### Synthesis of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Salt Form A)

### Preparation 1

### a) (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride¹

(R)-(5-Chloromethyl-isoxazol-3-yl)-cyclohexyl-phenyl-methanol (Intermediate 2) (3.00 g) and (R)-3-(3-fluoro-phenoxy)-1-aza-bicyclo[2.2.2]octane (Intermediate 1) (2.17 g) were mixed in acetonitrile (60 mL) and heated at 50° C. for 2 h. The reaction mixture was evaporated in vacuo and purified by silica gel chromatography (eluting with 1-15% methanol in DCM) to give the title compound as a white foam. This was dissolved in boiling acetonitrile (500 ml) and allowed to cool slowly to room temperature. The resulting white crystals were collected by filtration and dried in vacuo to give the title compound (3.9 g, 75%). ¹H NMR (400 MHz, DMSO-d6): δ 7.49 (dd, 2H), 7.40-7.29 (m, 3H), 7.25-7.20 (m, 1H), 6.93-6.79 (m, 4H), 5.90 (s, 1H), 4.96 (s, 1H), 4.77 (s, 2H), 3.95 (dd, 1H), 3.49 (d, 4H), 2.43 (s, 1H), 2.26-2.10 (m, 2H), 2.07-1.98 (m, 1H), 1.95-1.82 (m, 2H), 1.69 (d, 1H), 1.59 (s, 4H), 1.28-1.14 (m, 3H), 1.10-0.98 (m, 3H). LCMS (Method 1, 8.70 min). M⁺=491. ¹An alternative preparation of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride is described WO 2008/099186

### b) (R)-1-[3-((R)-cyclohexyl-hydroxy-phenyl-methyl)-isoxazol-5-ylmethyl]-3-(3-fluorophenoxy)-1-azonia-bicyclo[2.2.2]octane 2-hydroxy-ethanesulfonate (Salt Form A)

A solution of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane chloride (3.2 g) in warm DCM (50 ml) and methanol (0.5 ml) was stirred briskly and treated with a solution of ammonium isethionate (5 g) in water (20 ml). The reaction mixture was stirred at room temperature for 1 h, then cooled to 0° C. and stirred for 0.5 h. The resulting white precipitate was collected by filtration and washed with water and ether and dried in vacuo. The precipitate was dissolved in boiling acetonitrile (172 ml). The resulting solution was filtered whilst hot, and allowed to cool slowly to room temperature whilst being stirred. After 2 h, the resulting white crystals were collected by filtration and dried in vacuo to give the title compound (3.07 g, 82%). ¹H NMR δ (ppm) (DMSO-d₆): 7.47-7.42 (2H, m), 7.35-7.25 (3 H, m), 7.21-7.13 (1H, m), 6.81 (4H, d, J=43.75 Hz), 5.84 (1H, s), 4.92 (1H, s), 4.70 (2 H, s), 4.40 (1H, t, J=5.72 Hz), 3.90 (1H, dd, J=13.18, 8.10 Hz), 3.58 (2H, td, J=6.74, 5.72 Hz), 3.48-3.29 (5H, m), 2.56 (2H, t, J=6.74 Hz), 2.39 (1H, s), 2.21-2.04 (2H, m), 2.03-1.94 (1H, m), 1.93-1.77 (2H, m), 1.64 (1H, d, J=10.36 Hz), 1.54 (3H, d, J=9.07 Hz), 1.24-1.10 (3H, m), 1.10-0.93 (3H, m). LCMS (Method 1, 8.72 min). M⁺=491.

### Synthesis of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Salt Form A)

### Preparation 2

To a stirred suspension of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride² (155.83 g) and DCM (2380 mL) in a 5 L flask equipped with an overhead stirrer was added MeOH (23.8 mL) in one portion. After stirring for a few minutes a solution formed. To the stirred solution of the chloride salt was added a solution of isethionic acid, ammonium salt (61.60 g) in water (945 mL) over 5 minutes. The resulting two-phase reaction mixture was stirred vigorously and after a few minutes some seed crystals of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate were added. A few more were added after a further 35 minutes of stirring. Traces of solid formation were observed around the sides of the flask. It was stirred at room temperature for a further 2.5 hours and a dense precipitate began to form. Examination of a small aliquot of the reaction mixture under a microscope showed crystalline material. The stirred reaction mixture was cooled in an ice bath (with internal temperature 4° C. for 35 minutes). The solid became more granular. The solid was collected by filtration and washed with cold water (total volume 3.1 L in 400-60 mL portions) and then with ether (5×500 mL). It was sucked dry in air and then dried in vacuo at 40° C. overnight and then for a further 6 hours to give the product as a white crystalline solid (152.48 g). LC-MS (Method 2): Rₜ 8.91 min, m/z 491 [M]⁺. Purity>99%. ²A preparation of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride is described WO 2008/099186

The product (152.48 g) was then dissolved with stirring in refluxing IMS (2.8 L) and the hot solution was filtered. This solution was kept hot and stirred in a 10 L heated jacket reactor whilst the remaining material (151.64 g) was dissolved in refluxing IMS (2.8 L) and then filtered hot. The two solutions were combined in a 10 L heated jacket reactor and stirred and refluxed. A small amount of material had started to crystallise out, so further IMS (350 mL) was added until a solution formed. The stirred solution (stirring speed 88-89 rpm) was gradually allowed to cool [78° C. (reflux temperature) to 76.5° C. (internal temperature) over about 1 h and then 76.5-20° C. (internal temperature) over 4.5 hours and then stirred at 20° C. overnight]. Seed crystals were added to the stirred solution at 77° C., 69° C. and 59° C. Solid material had begun to crystallise out at base of reactor. More crystallisation was observed over the next few minutes as the mixture cooled down further. After stirring overnight the solid was collected by filtration, washed with cold IMS (˜300 ml) and dried by suction in air (for 2.5 hours) and then in vacuo at 40° C. overnight to give crystalline (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (274.48 g). LC-MS (Method 2): Rₜ 8.84 min, m/z 491[M]⁺. Purity>99%.

### Solid State Analysis of Salt Form A of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate

Instrument Details

X-Ray Powder Diffraction (XRPD)—PANalytical X'Pert machine in 2Ø-Ø configuration or a PANalytical Cubix machine in Ø-Ø configuration over the scan range 2° to 40° 2Ø with 100-second exposure per 0.02° increment. The X-rays were generated by a copper long-fine focus tube operated at 45 kV and 40 mA. The wavelength of the copper X-rays was 1.5418 Å. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.

Differential Scanning calorimetry (DSC) thermograms were measured using a TA Q1000 Differential Scanning calorimeter, with aluminium pans and pierced lids. The sample weights varied between 0.5 to 5 mg. The procedure was carried out under a flow of nitrogen gas (50 ml/min) and the temperature studied from 25 to 300° C. at a constant rate of temperature increase of 10° C. per minute.

Gravimetric Vapour Sorption (GVS) profiles were measured using a Surface Measurements Systems Dynamic Vapour Sorption DVS-1 or a DVS Advantage instrument. The solid sample ca. 1-5 mg was placed into a glass vessel and the weight of the sample was recorded during a dual cycle step method (40 to 90 to 0 to 90 to 0% relative humidity (RH), in steps of 10% RH).

### Solid Form Properties of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Salt Form A)—Preparation 1

Salt Form A prepared by preparation 1 was analysed by XRPD, GVS and DSC. The melting temperature was determined by DSC and found to have a sharp melt onset at approximately 214° C. (±2° C.). GVS determination gave no mass increase at 80% RH. An XRPD spectrum of ‘Salt Form A’ prepared in preparation 1 is presented in FIG. 1.

### Solid Form Properties of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate (Salt Form A)—Preparation 2

Salt Form A prepared by preparation 2 was analysed by XRPD, GVS and DSC. The melting temperature of Form A as determined by DSC was found to be 213° C. (onset) (±2° C.). GVS determination gave a weight increase of 0.15% at 80% RH (±0.3%). An XRPD spectrum of ‘Salt Form A’ prepared in preparation 2 is presented in FIG. 2.

### Salt Form Properties of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride (Comparative Example)

A sample of (R)-1-[3-((R)-Cyclohexyl-hydroxy-phenyl-methyl)-isoxazol-5-ylmethyl]-3-(3-fluoro-phenoxy)-1-azonia-bicyclo[2.2.2]octane chloride prepared in preparation 1 (step a) was analysed by XRPD, GVS and DSC. The melting temperature was determined by DSC and found to have a broad endothermic event (melt) onset at approximately 134° C. (±2° C.). GVS determination produced a mass increase of approximately 5% 1^{st} cycle and 6.5% 2^{nd} cycle. An XRPD spectrum of the chloride salt is presented in FIG. 3.

### Biological Activity of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate

Biological Activity of Muscarinic Antagonists

The inhibitory effect of (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate was determined by a Muscarinic Receptor Radioligand Binding Assay. Recombinant human M3 receptor was expressed in CHO-K1 cells. Cell membranes were prepared and binding of [3H]-N-methyl scopolamine ([3H]-NMS) and compounds was assessed by a scintillation proximity assay (SPA). The incubation time was 16 hours at ambient temperature in the presence of 1% (v/v) DMSO. The assay was performed in white 96 well clear-bottomed NBS plates (Corning). Prior to the assay, the CHO cell membranes containing M3 receptor were coated onto SPA WGA (Wheat germ agglutinin) beads (GE Healthcare). Non specific binding was determined in the presence of 1 μM Atropine. Radioactivity was measured on a Microbeta scintillation counter (PerkinElmer) using a 3H protocol with a 2 minutes per well read time. Compound inhibition of [3H]-NMS binding was determined typically using concentrations in the range 0.03 nM to 1 μM and expressed as percent inhibition relative to the plate specific radioligand binding for the plate. (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate exhibited a potency (as a Ki value) in the M3 binding assay of 0.66 nM,

1. A salt being (R)-1-[3-((R)-cyclohexyl-hydroxy-phenyl-methyl)-isoxazol-5-ylmethyl]-3-(3-fluoro-phenoxy)-1-azonia-bicyclo[2.2.2]octane 2-hydroxy-ethanesulfonate.

2. A salt according to claim 1, which exhibits at least the following characteristic X-ray powder diffraction peaks (expressed in degrees 2θ when using λ=1.5418): 8.4, 14.7 and 16.8.

3. A salt according to claim 2 having an X-ray powder diffraction pattern substantially the same as that shown in FIG. 1.

4. A pharmaceutical composition comprising a salt according to any one of claims 1 to 3 in association with a pharmaceutically acceptable adjuvant, diluent or carrier.

5. A salt according to any one of claims 1 to 3 for use in therapy.

6. Use of a salt according to any one of claims 1 to 3 in the manufacture of a medicament for use in treating chronic obstructive pulmonary disease.
